(19) United States
(12) Patent Application Publication
(10) Pub. No.: US 2015/0118210 A1
(43) Pub. Date: Apr. 30, 2015

(54) COMPOSITION FOR ENHANCING CELL ENGRAFTMENT AND HOMING PROPERTIES CONTAINING PRP AS ACTIVE INGREDIENT

(71) Applicant: SNU R&DB FOUNDATION, Seoul (KR)

(72) Inventors: Hyun Chul Jo, Seoul (KR); Hyang Kim, Seoul (KR)

(73) Assignee: SNU R&DB FOUNDATION, Seoul (KR)

(21) Appl. No.: 14/403,711

(22) PCT Filed: May 28, 2013
(86) PCT No.: PCT/KR2013/004642

§ 371 Date: Nov. 25, 2014

(30) Foreign Application Priority Data
May 29, 2012 (KR) 10-2012-0057008

(51) Int. Cl. A61K 35/19; C12N 5/0775

(52) U.S. Cl. CPC A61K 35/19; C12N 5/0663; C12N 2500/84
USPC 424/93.72; 435/325

(57) ABSTRACT

The present invention provides a composition for enhancing cell engraftment and homing properties, containing platelet-rich plasma (PRP) as an active ingredient. The present invention provides a method for improving cell engraftment and homing properties, comprising the step of treating in vitro isolated cells with a composition containing PRP as an active ingredient. According to the present invention, when a cell to be transplanted or a region to which cells are transplanted is treated with PRP, or a cell to be transplanted is mixed with PRP, transplantation to an in vivo region is carried out, thereby increasing cell engraftment in a transplanted region and increasing cell homing from a transplanted region to a damaged region.

### BACKGROUND OF INVENTION

1. Field of the Invention

The present disclosure relates to a composition comprising platelet derived plasma for enhancing cells' attachment and homing properties.

2. Description of the Related Art

Recently cell therapy in which cells or cellular materials are used for treating certain disease has become a new paradigm in medicine. For example, differentiated tissue specific cells such as chondrocytes, myoblasts, osteoblasts, cardiocytes, fibroblasts, adipocytes, neurons, hepatocytes and undifferentiated stem cells or precursor cells such as hematopoietic stem cells, mesenchymal stem cells, tissue specific progenitor cells, embryonic stem cells, induced pluripotent stem cells are used as a medicine in various diseases.

However, the most important and common problems encountered in the clinical application of the cells reside in the cells' low abilities for homing meaning finding damaged sites or tissues, and engraftment as well as the cells low survival rate due to anoikis of the damaged tissues or organs and unfavorable microenvironment (13). Particularly, it is known that homing receptor such as CXCR4 which is a receptor for SDF-1 is not found in cultured mesenchymal stem cells and thus their homing ability is very low compared to those found in leukocytes and hematopoietic stem cells (14).

Therefore it is important in a successful implementation of cell therapy that cells are efficiently targeted and engrafted to the site of interest. Particularly, cells with increased homing activity can remarkably reduce the number of cells required for cell therapy as well as it can reduce the time, cost, and efforts needed for culturing cells. Further it can provide a better prognosis for patient.

Previously known methods for improving homing and engrafting activities of cells are: 1) delivering genes encoding homing receptors such as CXCR4 (15, 16); 2) chemically engineering glycans on the cell surface (17); 3) conjugation of antibodies through bispecific antibodies or palmitated protein G or A (18) and 4) controlling conditions for cell culture to promote the expression of particular homing receptors.

Throughout the present disclosure, numerous papers and patent documents are referred to, the contents of which are incorporated herein by reference in its entirety to more clearly describe the present invention and the status of the related art.

### DETAILED DESCRIPTION OF THE INVENTION

### Problems to Be Solved

The present disclosure is to provide a composition for enhancing or increasing or improving cells' homing and engraftment activity.

Also, the present disclosure is to provide a method for enhancing or increasing or improving cells' homing and engraftment activity by treating isolated cells with the present composition.

These and/or other aspects and advantages of the invention will become apparent and more readily appreciated from the following description of the present disclosure and claims.

### SUMMARY OF THE INVENTION

In one aspect the present disclosure provides a pharmaceutical composition for enhancing or increasing or improving cells' homing and engraftment activity comprising platelet rich plasma (PRP). The PRP of the present disclosure may be comprised in an amount to achieve the desired effects according to the present disclosure and may vary depending on the type of cells treated, its biochemical properties and the site to which it is applied. In one embodiment, the present PRP comprise 2×10⁵ to 3×10⁸ platelets per microliter of plasma.

The present composition can be advantageously used for restoration and/or for therapy for muscloskeletal injury, lesion, or disease.

In one embodiment, the present composition is used for cells particularly for stem cells, for example multipotent stem cells for example, multipotent mesenchymal stem cells, multipotent hematopoietic stem cells, multipotent neural stem cells, multipotent endothelial stem cells, multipotent epithelial stem cells, multipotent epidermal stem cells, induced pluripotent stem cells, multipotent muscle stem cells, multipotent adult stem cells, multipotent pancreatic stem cells, multipotent cardiac stem cells, multipotent vascular stem cells, multipotent follicular stem cells, multipotent corneal stem cells, multipotent adipose stem cells, multipotent umbilical cord/umbilical cord blood stem cells, multipotent tooth stem cells, multipotent liver stem cells, and multipotent cancer stem cells, but the cells are not limited thereto.

In one embodiment, the present composition is used to pretreat isolated cells ex vivo to enhance or increase or improve the cells' homing and engraftment activity before the cells are transplanted to the site of interest in vivo.

The present composition is applied to the site of interest in the body alone or in combination with the cells to be transplanted.

In other aspect, the present disclosure also provides methods for improving, enhancing or increasing the cells' homing and engraftment activity comprising treating isolated cells with the present composition or applying the present composition to the site of interest in the body to which the isolated cells are transplanted.

In a further aspect, the present disclosure also provides platelet rich plasma which is used for improving, enhancing or increasing the cells' homing and engraftment activity.

The foregoing summary is illustrative only and is not intended to be in any way limiting. Additional aspects and/or advantages of the invention will be set forth in part in the description which follows and, in part, will be obvious from the description, or may be learned by practice of the invention.

### Advantageous Effects

The advantageous effects of the present disclosure are summarized as below:

(a) The present disclosure provides a composition comprising PRP as an effective ingredient that increases, enhances or improves cells' homing and engraftment activity,

(b) When the present composition is used to pretreat isolated cells in vitro or to pretreat the site of interest in the body to which the cells are to be transplanted alone or in combination with cells to be transplanted, the cells' homing and engraftment activity is enhanced, increased or improved.

(c) The present disclosure also provides methods for improving, enhancing or increasing cells' homing and engraftment activity comprising treating the isolated cells ex vivo with the present composition.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is results from H&E staining of osteochondral plugs showing the histological differences depending on the severity of the injuries. The severity of the damage was assessed as the following grades: 0, normal; 1, thickness of the cartilage is reduced to less than 50% at the lesion; 2, thickness of the cartilage is reduced to more than 50% at the lesion; 3, subchondral osseous lamina is exposed at the lesion.

FIG. 2 is a schematic representation of the experiment using osteochondral plugs which are disposed on a 2% agarose gel.

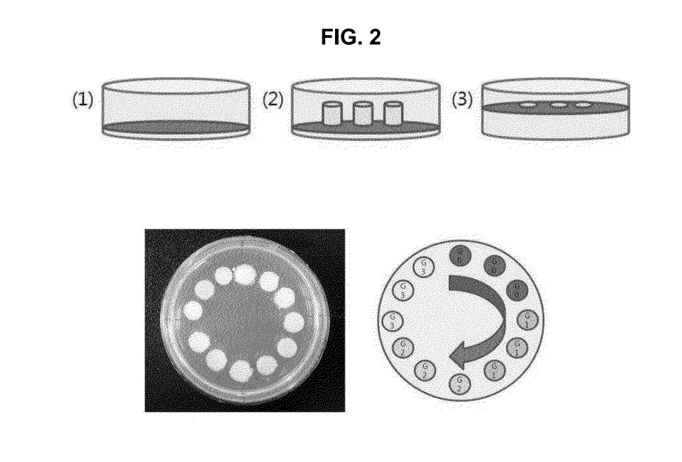

FIG. 3 is a schematic representation of the experimental design regarding ex vivo homing and engraftment assay.

FIG. 4 is micrographs of bone marrow mesenchymal stem cells engrafted on a plastic culture dish.

FIG. 5 is a graph showing the effect of the present PRP on the engraftment of bone marrow MSCs.

FIG. 6 is micrographs of chondrocytes engrafted on a plastic culture dish.

FIG. 7 is a graph showing the effect of the present PRP on the engraftment of chondrocytes.

FIG. 8 is results from the ex vivo experiments performed as in FIG. 2 showing the effect of the present PRP on the engraftment and homing of bone marrow MSCs.

FIG. 9 is confocal microscopic results showing the effect of the present PRP on the engraftment and homing of bone marrow MSCs ex vivo.

### DETAILED DESCRIPTION OF THE EMBODIMENT

The present disclosure is based on the findings that when the cells are treated with PRP, their homing activity and engraftment is very effectively increased, enhanced or improved.

In one embodiment, the present disclosure is related to a composition comprising PRP as an effective ingredient which is used to improve, enhance, or increase cells' homing and engraft activity.

Platelets comprises in theirs granules various bioactive materials including anti-protein proteinases, proteinases, engraftment proteins, chemokines, cytokines, growth factors, inflammatory factors related to tissue therapy and regeneration which includes three steps, i.e., inflammatory phase, trophic phase and remodeling phase (1-3). The inflammatory phase continues for about 2-3 days after injuries, and is characterized by extravasation and accumulation of leukocytes and activation of monocytes/macrophages. Neovascularization is started to provide bloods to the new tissues and thus to promote the metabolism and the endothelial cells are activated at the trophic or anabolic stages to begin the vascular formation. Cells migrate and divide using fibrin matrix as a support and differentiate into cells that produce collagen, proteoglycan and extracellular matrix. At the remodeling phase, the density of cells is decreased, and the whole metabolism at the injured tissue is decreased. The activation of a signal transduction activity specific for each phase limits the duration time of each phase and each phase is silenced or balanced by endogenous signals promoting the progression into the next phase.

PRP(Platelet-Rich Plasma) is a concentrate of platelet and comprises at least about 1,000×10³ platelets per microliter, and comprises 3 to 5 times more platelets compared to whole blood (4). PRP can secret bioactive materials in granules at the concentration higher than the physiological level (5). Also, PRP has a property of reconstructing or regenerating damaged region in blood vessels, thus it is known to be effective for treating arthritis, chronic lumbodynia, pelycalgia, shoulder or knee ligament injuries as stimulants to promote regeneration of tissues for prolotherapy or for regenerating skin. Initially PRP was widely used for maxillofacial and plastic surgery (4), and at present, PRP is used for promoting or increasing the efficacy of various tissue therapy regeneration including bone (6), cartilage (7), tendon (8), ligament (9) and muscles (10,11). However, the mechanism of its action and tissue therapy is not known (12).

The term “PRP (platelet rich plasma)” refers to platelet concentrates the level of which is higher than that is generally found in bloods. For example, the concentration of PRP may be at least 2 time, 5 times, 10 times, 100 times or more compared to the normal level found in blood.

PRP may comprise components other than platelets. In PRP, the platelets may be comprised in PRP as at least 50%, at least 75%, at least 95%, at least 99%. Components other than platelets which may be comprised in PRP include plasma, growth factors, leucocytes and/or other blood components. The origin of PRP may be autologous, or allogeneic. Or PRP may be obtained from sources of platelets and/or plasma. Preferably, PRP is obtained from autologous plasma. PRP may be obtained from various animals, particularly human.

In the present disclosure, PRP may be obtained using methods known in the related art. The method may further comprise a step to treat PRP when or before it is used to change its physical property such as treatment with thrombin and calcium, or collagen, or treatment with snake venom such as batroxobin to transform fibrinogen to fibrin by activation or gelation of PRP.

In one embodiment, the present PRP comprises about 2×10⁵-3×10⁸ platelets per microliter of plasma, particularly about 1×10⁶-1×10⁷ platelets per microliter of plasma, more particularly about 1×10⁶-5×10⁶ platelets per microliter of plasma.

The term “attachment” refers to an ability of cells effectively to adhere or engraft themselves to the site of implant or transplant and includes “engraftment”, “engrafting activity”, or “engrafting capacity” Also enhancing, improving or increasing attachment or engraftment refers that the efficiency of cell transplant or implant are improved, increased or enhanced resulting in the reduced graft rejection and/or increased adherence of cells to the site of implant of interest.

The term “homing” or “homing activity” refer to an ability of cells to effectively migrate from the site of implant to the damaged site.

According to one embodiment of the present disclosure, when the present composition are used to treat cells in a culture dish, or cells are pretreated with the present composition before being seeded onto a dish, the engraftment of the cells are markedly increased. Or when the cells that are pretreated with the present composition are applied to the damaged site in the body, the migration of cells to the damaged site is increased.

The present composition may be applied to a variety of cells which may be used for cell transplant. For the cells which may be used include but are not limited to stem cells, liver cells, pancreatic cells/pancreatic blastoma, kidney cells, thyroid cells, lung cells, myocardiocytes, myocytes/myoblasts/myosatellite cells, osteocytes/osteoblasts/osteoclasts, chondrocytes/chondroblasts, tendon cells, ligament cells, synovial cells, fibroblast, skin cells, endothelial cells, blasts, hematopoietic cells, germ cells, adipocytes, epithelial cells, dermal cells, follicular cells/keratinocytes/ hair matrix cells, neural cells/neuroblasts, immune cells, cornea cells or retinal cells. In one embodiment, the present composition is applied to stem cells or chondrocytes.

In one preferred embodiment, the cells to which the present composition is applied is multipotent stem cells, particularly, multipotent mesenchymal stem cells, multipotent hematopoietic stem cells, multipotent neural stem cells, multipotent endothelial stem cells, multipotent epithelial stem cells, multipotent epidermal stem cells, induced pluripotent stem cells, multipotent muscle stem cells, multipotent adult stem cells, multipotent pancreatic stem cells, multipotent cardiac stem cells, multipotent vascular stem cells, multipotent follicular stem cells, multipotent corneal stem cells, multipotent adipose stem cells, multipotent umbilical cord/umbilical cord blood stem cells, multipotent tooth stem cells, multipotent liver stem cells, and multipotent cancer stem cells. In one preferred embodiment the cell is multipotent MSCs.

In one preferred embodiment, the present composition improves increases or enhances the regeneration of cells at the damaged site. The treatment by the present composition results in the increased homing activity of the cells to the damaged site and thus the regeneration of cells or tissues at the damaged site is increased by the cells migrated to the damaged sites.

According to one embodiment, the present composition is used to pretreat isolated cells ex vivo to improve, increase or enhance engraftment and homing activity of the cells. As evident as described in Example below, when the ex vivo isolated cells were pretreated with the present composition, it was found that the cells ability to engraft were markedly increased.

According to one preferred embodiment, the isolated cells ex vivo, includes stem cells, liver cells, pancreatic cells/pancreatic blastoma, kidney cells, thyroid cells, lung cells, myocardiocytes, myocytes/myoblasts/myosatellite cells, osteocytes/osteoblasts/osteoclasts, chondrocytes/chondroblasts, tendon cells, ligament cells, synovial cells, fibroblast, skin cells, endothelial cells, blasts, hematopoietic cells, germ cells, adipocytes, epithelial cells, dermal cells, follicular cells/keratinocytes/ hair matrix cells, neural cells/neuroblasts, immune cells, cornea cells or retinal cells. In one embodiment, the present composition is applied to stem cells or chondrocytes. In one preferred embodiment, the cells are pretreated for about 5 min to 3 hrs, more particularly about 10 min to 2 hrs, most particularly for 10 min to 30 min.

The term “ex vivo” refers to a process to isolate cells from the subject and manipulate the cells outside of the subject (for example, in vitro).

According to one preferred embodiment, the present composition also can be applied to the site of transplant. When the present composition is applied to the site of transplant before the cells are applied, the migration of cells to the site of implant and the cells engraftment are effectively induced.

According to one preferred embodiment, the present composition can be applied to the site of implant in combination with the cells to be transplanted. When the cells are pretreated with the present composition before it being implanted to the site of interest, the engraftment of the cells is increased due to the increased adherence activity provided by the present composition.

According to one preferred embodiment, the cells that are used for the implant include stem cells, liver cells, pancreatic cells/pancreatic blastoma, kidney cells, thyroid cells, lung cells, myocardiocytes, myocytes/myoblasts/myosatellite cells, osteocytes/osteoblasts/osteoclasts, chondrocytes/chondroblasts, tendon cells, ligament cells, synovial cells, fibroblast, skin cells, endothelial cells, blasts, hematopoietic cells, germ cells, adipocytes, epithelial cells, dermal cells, follicular cells/keratinocytes/ hair matrix cells, neural cells/neuroblasts, immune cells, cornea cells or retinal cells. In one embodiment, the present composition is applied to stem cells or chondrocytes. In one embodiment, the present composition is applied to stem cells or chondrocytes.

According to one preferred embodiment, the cells treated with the present composition have an increased homing activity to the damaged site. According to the present disclosure, when the cells which were pretreated with the present composition were implanted to osteochondral pieces, it was found that the cells migrated to the surface of osteochondral piece in which the damage and degenerated changes had been developed. Particularly, migration to the site which was most severely damaged was increased.

In other aspect, the present disclosure relates to methods for increasing cells abilities for attachment or homing. The present method comprises a step of treating ex vivo isolated cells and/or in vivo site to which the cells are to be transplanted with the present composition. The present method further comprises a step of implanting the cells treated with the present composition to the site of interest where the cell therapy is required. The ex vivo isolated cells are as described hereinbefore.

The present disclosure is further explained in more detail with reference to the following examples. These examples, however, should not be interpreted as limiting the scope of the present invention in any manner.

### EXAMPLES

### Materials and Methods

### Mesenchymal Stem Cells

Human MSCs were obtained using a standard procedure from bone marrow as described below. Ficoll-Paque™ PREMIUM was used to isolated monocytes by centrifugation. Then the cells were washed twice and suspended in DMEM-LG (HyClone, Thermo Fisher Scientific Inc., Waltham, Mass., USA) containing 10% bovine (HyClone, Thermo Fisher Scientific Inc., Waltham, Mass., USA) supplemented with 100 U/ml penicillin /100 μg/ml streptomycin (HyClone, Thermo Fisher Scientific Inc., Waltham, Mass., USA) and seeded at the concentration of 1×10⁶ cells/cm² and incubated at 37° C., in a 5% CO² incubator with humidity. The medium was changed at one or two days after the incubation and after than the medium was changed every 3 days. When the cells reached 80% confluence, the cells were passaged at the ratio of 1:3.

### Chondrocytes

Chondrocytes were washed with PBS(phosphate buffered saline, DPBS) without calcium and magnesium and ground therein. Then, DMEM(high-glucose Dulbecco's Modified Eagle's Medium, Life Technologies, Rockville, Md.) containing 100 U/mL penicillin, 100 mg/ml streptomycin 0.25 mg/ml amphotericin B (Life Technologies) supplemented with 0.6% collagenase (Sigma, St. Louis, Mo., USA) was added for 6 hrs at 37° C. to release chondrocytes. Then nylon mesh with 100 nm size (BD, Franklin Lakes, N.J., USA) was used to remove chondrocytes that was not ground and the filtered material was then centrifuged to obtain chondrocytes which were then washed twice. The cells were then resuspended in DMEM comprising 10% Fetal bovine serum (FBS, Life Technologies) supplemented with 25 mg/ml L-ascorbic acid (Sigma), 100 U/ml penicillin /100 μg/ml streptomycin (HyClone, Thermo Fisher Scientific Inc., Waltham, Mass., USA) and seeded onto a 10 mm culture dish and incubated at 37° C., in a 5% CO² incubator with humidity for 7 days. The medium was changed at one or two days after the incubation and after than the medium was changed every 3 days. When the cells reached 80% confluence, the cells were passaged at the ratio of 1:3. The cells were treated with trypsin-EDTA solution (0.25% trypsin, 0.53 mM EDTA; Life Technologies) for 5 min to detach the cells from the dish and washed twice with the culture medium and suspended in PBS or PRP. Cells passaged 2-5 times were used for the experiment.

### Preparation of PRP

PRP was collected from subjects according to a standard procedure using a platelet isolation and releasing system having a leukocyte removal kit (COBE spectra LRS Turbo; Caridian BCT, Lakewood, Colo., USA)(20). The final concentration of platelet was 1,400×10³ per microliter. As an anticoagulant agent, saline and ACDA(anti-coagulant acid citrate dextrose) were prepared according to manufacturer's instruction. The complete blood counts were counted. The platelet concentration of PRP was adjusted to 1,000×10³ per microliter for the experiments and diluted or concentrated as needed. To activate PRP, 10% calcium gluconic acid salt comprising 166.7 IU/ml of thrombin (Reyon Pharmaceutical, Korea) was added to the PRP at 1:10 vol/vol ratio. The controls were treated with saline only.

### In Vitro Homing and Engraftment Assay

Cells were resuspended in saline (controls) or treated with PRP before it being seeded onto a plate as follows: Controls. Suspended in saline; PRP, suspended in PRP; Pretreated with PRP for 10 min (pretreatment means suspending cells with PRP before the transplant), cell were pretreated with PRP for 10 min before the transplant; Pretreated with PRP for 30 min, cell were pretreated with PRP for 30 min before the transplant. Cells were seed onto each well of a 48 well plate and incubated at 37° C. in a 5% CO2 incubator with humidity for 2.5, 5, 10, 15, 20, 30 and 60 min. Each well was washed twice to remove unattached cells after the incubation. The attached cells were counted using an image processing program.

### Ex Vivo Homing and Engraftment Assay.

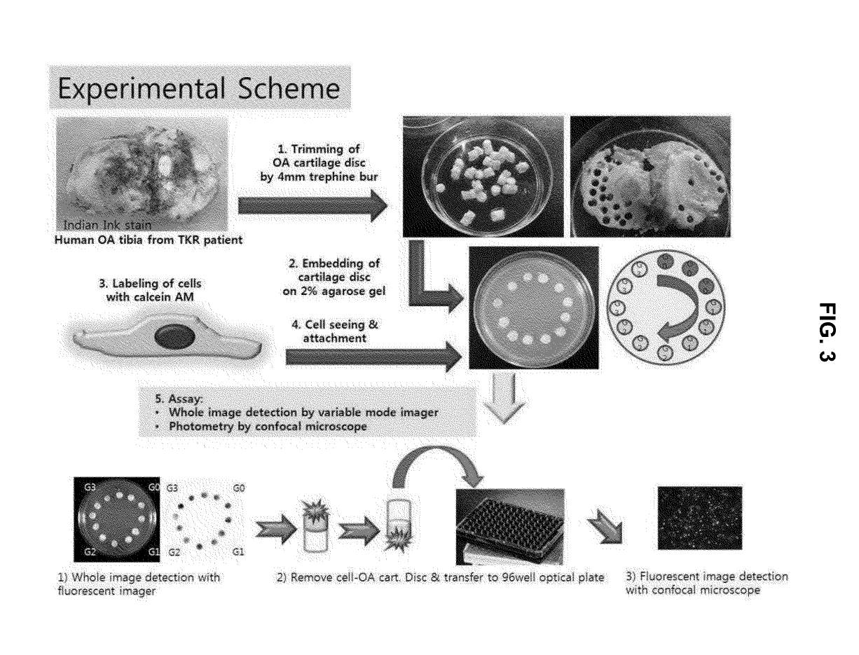

Experimental design was as depicted in FIG. 3.

### Osteochondral Disc Preparation

Osteochondral block was prepared using the method known in the art with the following minor modification from proximal tibia derived from a patient who has undergone knee joint replacement surgery. The surface of proximal tibia was examined and assessed according to the grades set by International Cartilage Repair Society, ICRS. .The grades depending on the severity of the injury are as follows: 0, normal; 1, thickness of the cartilage is reduced to less than 50% at the lesion; 2, thickness of the cartilage is reduced to more than 50% at the lesion; 3, subchondral osseous lamina is exposed at the lesion.

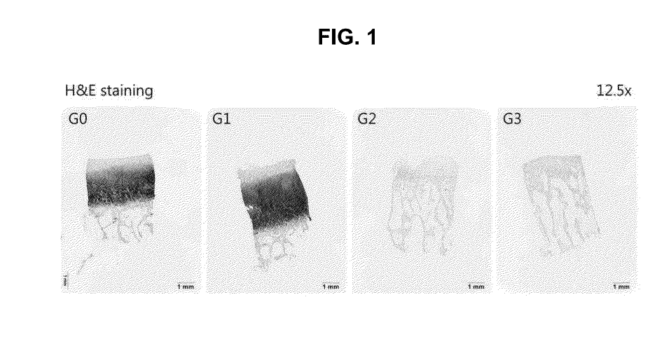

Osteochondral plugs were prepared as a cylindrical shape (diameter: 4.0 mm, height: 4.0 mm) using a Trephine burr. The histological characteristics of the surface were as shown in FIG. 1. The plugs were kept at −80° C. and thawed at RT one hour before use in DPBS. The variability in the assessed grades was adjusted by several assessments (two time assessments when the samples were received and confirmed again when the discs were prepared and ready for use for the experiments). Osteochondral plugs were implanted onto a 35 mm culture dish using 2% agarose gel which was prepared in 0.5% TBE buffer and poured to the dish containing the plugs. The osteochondral plugs were disposed towards the upper side. The grade 0 plugs were disposed on the upper right side, grade 1 plugs on the lower right side, grade 2 plugs on the lower left side; and grade 3 on the upper left side.

### Homing and Engraftment Assay Using Osteochondral Disc

Mesenchymal stem cells from bone marrow was detached from the cell culture dish and washed twice with DPBS and suspended in DEME-LG at the concentration of 5×10⁶ cells/ml. Then 1 mM Calcein AM stock was added and mixed and incubated for 30 min at 37° C. The cells were then washed twice with DPBS and suspended either with saline (controls) or treated with PRP as follows: Controls. Suspended in saline; PRP, suspended in PRP; Pretreated with PRP for 10 min (pretreatment means suspending cells with PRP before the transplant), cell were pretreated with PRP for 10 min before the transplant; Pretreated with PRP for 30 min), cell were pretreated with PRP for 30 min before the transplant. Cells were then added to the culture dish containing the plugs as described above at the concentration of 1.0×10⁴ cells/mm² and incubated for 15 min at 37° C. in a 5% CO₂ incubator with humidity. After the incubation, the dishes were washed four times and fluorescent images were taken using a scanner (typhoon 9410 scanner, GE Healthcare). The images were quantified using ImageQuant. The plugs were transferred to a 96 well plate with the surface being toward the bottom. The images of the cells were taken using a confocal microscope (Carl Zeiss, Germany). Images taken were captured using image analyzer (Carl Zeiss, Germany).

### Statistical Analysis

All data were expressed as average and standard deviation. Significant difference was assessed using T-test and one-way ANOVA. Dunnett test was used for the comparison of controls and Tukey's test was used for the comparison of PRP and pretreatment with PRP for 10 and 30 min. P<0.05 was considered statistically significant.

### Results

Assay of In vitro Homing and Engraftment

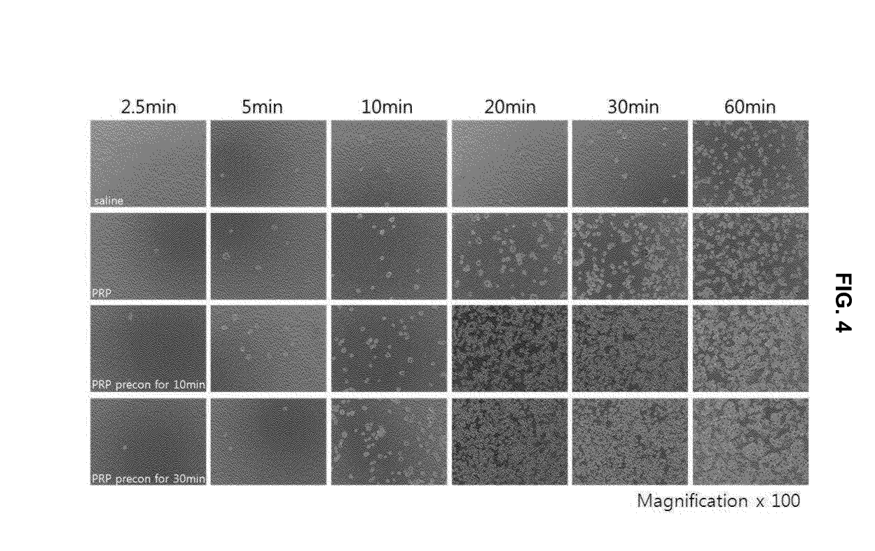

PRP increased the cell attachment regardless of the type of cells used in a time dependent manner as shown in FIGS. 4 to 7. Thus the cells' attachment was the lowest in the control samples and was the highest in the samples pretreated for 30 min and the PRP samples and samples pretreated for 10 min in between.

The attachment of bone marrow MSC was increased in a time dependent manner and the attachment was enhanced by treatment with PRP. The number of cells in control incubated for 60 min was similar to that of cells treated with PRP for 20 min. The cells pretreated with PRP for 10 min and 30 min was slightly higher than when the cells were incubated for 10 min. In controls, the percentage of attached cells was 57.11%. In contrast, the percentage of attached cells was not markedly different in 10 min and 30 min pretreated samples, and the plateau was reached at 15 min and 20 min after the incubation respectively (Refer to FIG. 4).

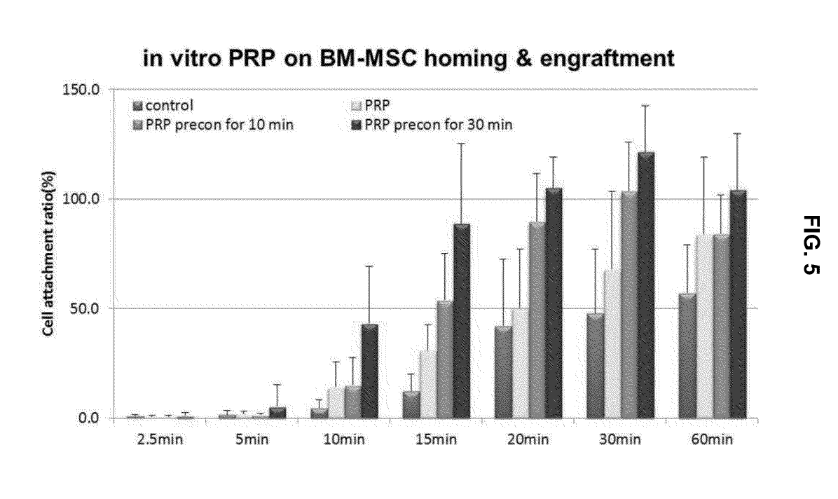

The attachment of cartilage cells was increased in a time dependent manner, and markedly increased by PRP. The attachment in control cells incubated for 60 min was similar to that incubated cells pretreated for 30 min with PRP. Cells pretreated for 10 min and 30 min with PRP was similar when the cells were incubated for 10 min and 20 min respectively. In control, the percentage of attached cells was 76.56%. In contrast, PRP treated cells and cells pretreated for 10 min reached a plateau at 60 min after the incubation and cells pretreated with PRP for 30 min reached a plateau at 30 min after the incubation (refer to FIG. 5).

### Bone Marrow Derived MSCs.

The results of comparison of the attachment of cells among the treated groups were shown in table 1, FIGS. 4 and 5. At 2.5 and 5 min, it was found that there was no significant difference among the tested groups. However, significant difference was found at 10, 15, 20, 30 and 60 min incubation time point.

TABLE 1
The comparison of the attachment of bone marrow MSCs
under various conditions.

| | | | 10 min | 30 min | |
|---|---|---|---|---|---|
| | | | PRP | PRP | p- |
| | Control | PRP | pretreatment | pretreatment | value |
| 2.5 | 0.94 ± 0.63 | 0.77 ± 0.35 | 0.74 ± 0.38 | 1.17 ± 1.45 | 0.57 |
| min | | | | | |
| 5 min | 1.72 ± 1.75 | 1.98 ± 1.23 | 1.62 ± 0.58 | 5.60 ± 9.74 | 0.17 |
| 10 min | 4.65 ± 3.79 | 14.37 ± 11.31 | 15.26 ± 12.35 | 43.24 ± 26.01 | 0.00 |
| 15 min | 12.37 ± 7.86 | 31.05 ± 11.71 | 53.88 ± 21.29 | 88.68 ± 36.79 | 0.00 |
| 20 min | 42.16 ± 30.46 | 50.48 ± 26.79 | 89.84 ± 21.93 | 105.02 ± | 0.00 |
| | | | | 14.11 | |
| 30 min | 47.83 ± 29.19 | 68.00 ± 35.57 | 103.91 ± | 121.50 ± | 0.00 |
| | | | 22.18 | 21.19 | |
| 60 min | 57.11 ± 22.08 | 83.88 ± 35.40 | 84.22 ± 17.52 | 104.21 ± | 0.00 |
| | | | | 25.74 | |

When the cells were incubated for 10 min, in the group that was pretreated for 30 min with PRP, the cells' attachment was significantly increased compared to other three groups. (In all cases, P<0.001). There was found no significant difference among the control, PRP, 10 min PRP pretreatment groups. At 15 min incubation condition, in the 10 min and 30 min PRP pretreatment groups, the cell attachment was significantly increased compared to the control (P=0.001 and <0.001, respectively). There was found no significant difference between PRP group and the controls (P=0.204), in 30 min PRP pretreatment group, significant difference was found compared to PRP and 10 min PRP pretreatment group (P<0.001 [symbol] 0.012, respectively). At 20 min incubation condition, significant difference was found in 10 and 30 min PRP pretreatment groups (in all cases P<0.001). There was found no significant difference between PRP group and the controls (P=0.734). At 30 min incubation condition, Significant difference was found in 30 min PRP pretreatment group compared to PRP group (P<0.001). At 30 min incubation condition, 10 min and 30 min PRP pretreatment groups showed significant difference in the attachment compared to the control (in all cases, P<0.001). However there was no significant difference between PRP group and the control (P=0.193). At 60 min incubation, PRP, 10 and 30 min PRP pretreatment groups showed significant difference in the attachment compared to the control (P=0.041, =0.381, [symbol] <0.001, respectively) and but there was found no significant difference among themselves.

### Cartilage Cells

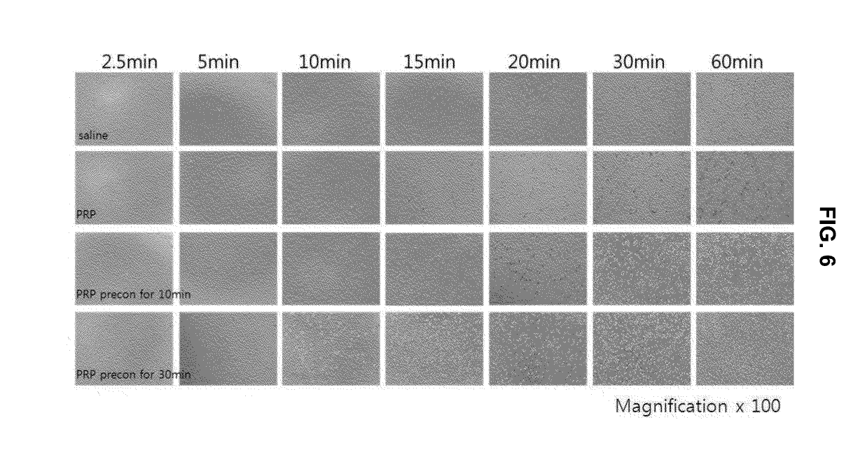

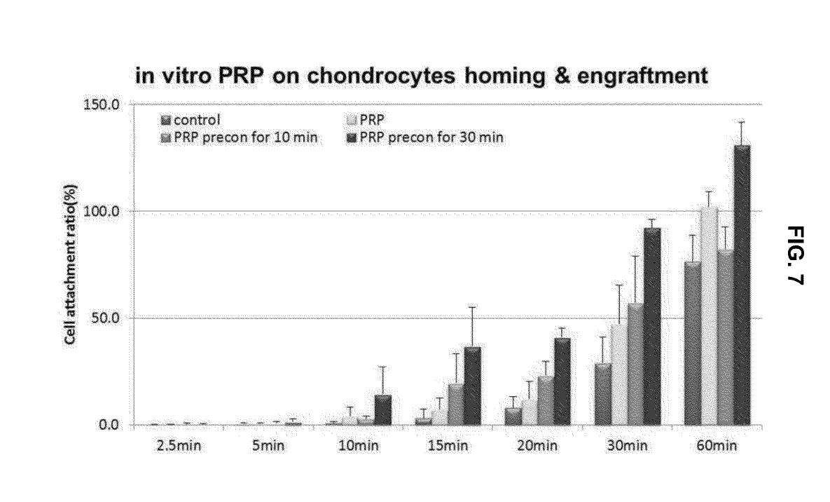

The results of comparison of the attachment of the cells among the groups at each time point were shown in table 2, FIGS. 6 and 7. At 2.5 and 5 min, it was found that there was no significant difference among the tested groups. However, the significant difference was found at 10, 15, 20, 30 and 60 min incubation time point.

TABLE 2
The comparison of the
attachment of cartilage cells under various conditions.

| | | | 10 min | 30 min | |
|---|---|---|---|---|---|
| | | | PRP | PRP | p- |
| | Control | PRP | pretreatment | pretreatment | value |
| 2.5 | 0.10 ± 0.12 | 0.30 ± 0.10 | 0.30 ± 0.54 | 0.41 ± 0.34 | 0.45 |
| min | | | | | |
| 5 min | 0.46 ± 0.40 | 0.71 ± 0.25 | 0.89 ± 0.69 | 1.50 ± 1.44 | 0.20 |
| 10 min | 1.01 ± 0.73 | 4.31 ± 4.04 | 3.02 ± 1.14 | 14.65 ± 12.47 | 0.01 |
| 15 min | 3.30 ± 4.02 | 7.48 ± 5.03 | 19.62 ± 13.89 | 36.89 ± 18.18 | 0.00 |
| 20 min | 8.16 ± 5.08 | 12.40 ± 8.08 | 23.22 ± 6.69 | 41.05 ± 4.28 | 0.00 |
| 30 min | 29.03 ± 12.24 | 47.13 ± 18.32 | 57.20 ± 21.91 | 92.31 ± 3.84 | 0.00 |
| 60 min | 76.56 ± 12.24 | 102.15 ± | 82.42 ± 10.40 | 130.95 ± | 0.00 |
| | | 7.06 | | 10.72 | |

At 10 min incubation condition, the attachment of cells in 30 min PRP pretreatment group was significantly increased compared to 10 min pretreatment group (P=0.005 [symbol] 0.029, respectively). There was found no significant difference between PRP group and 10 min PRP pretreatment group. At 15 min incubation condition, the attachment of cells in 30 min PRP pretreatment group was significantly different compared to the control and PRP group (P<0.001 and P=0.002, respectively). However no significant difference was found compared to 10 min PRP pretreatment group (P=0.088). At 20 min incubation time, the attachment of cells in 10 and 30 min PRP pretreatment group was significantly different compared to the control (P<0.001 and P=0.001 respectively). However, there was found no significant difference between PRP group and control (P=0.513). The 10 and 30 min PRP pretreatment groups was significantly different compared to PRP group (P=0.032 and P=<0.001, respectively). At 30 min incubation time, the 10 and 30 min PRP pretreatment groups were significantly different compared to control (P<0.001 and P=0.015, respectively). But there was found no significant difference between PRP group and control (P=0.142). The 30 min PRP pretreatment group was significantly different compared to PRP group and 30 min PRP pretreatment group P<0.001 and P=0.005, respectively). At 60 min incubation, PRP group and 30 min PRP pretreatment group showed significant difference compared to the control (P=0.001 and P=<0.001, respectively). In 30 min PRP pretreatment group, significant difference was found compared to PRP group and 10 min PRP pretreatment group (P=0.001 and P=<0.001, respectively).

### Ex Vivo Homing and Engraftment Assays

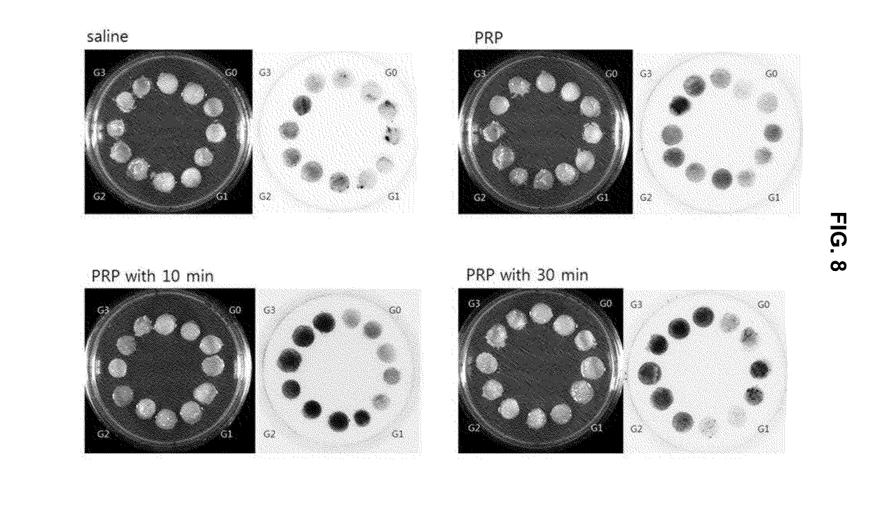

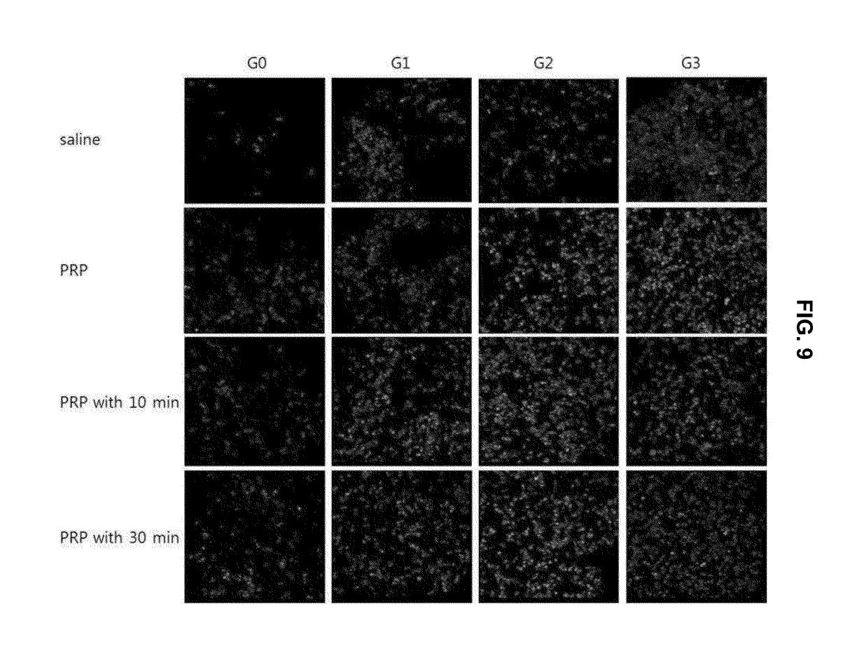

Bone marrow derived MSCs that were implanted were found to migrate the plugs and be engrafted as shown in FIGS. 8 and 9. The homing of the cells and engraftment were increased depending on the severity of the lesion and PRP treatments. In consistent with in vitro results, the engraftments were increased most at 30 min PRP pretreatment group followed by 10 min PRP pretreatment group and PRP group and the control. Homing and engraftment were increased depending on the severity of the lesion, which were further increased by treatment with PRP. In cartilages with grades 1 to 3 (G1 to G3), the homing and engraftment were increased with PRP treatment regardless of PRP pretreatment (FIG. 9).

With respect to the use of substantially any plural and/or singular terms herein, those having skill in the art can translate from the plural to the singular and/or form the singular to the plural as is appropriate to the context and/or application. The various singular/plural permutations may be expressly set forth herein for sake of clarity.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

### REFERENCES

1. Anitua, E., Andia, I., Ardanza, B., Nurden, P., and Nurden, A. T. 2004. Autologous platelets as a source of proteins for healing and tissue regeneration. Thromb Haemost 91:4-15

2. Alsousou, J., Thompson, M., Hulley, P., Noble, A., and Willett, K. 2009. The biology of platelet-rich plasma and its application in trauma and orthopaedic surgery: a review of the literature. J Bone Joint Surg Br 91:987-996.

3. Eppley, B. L., Pietrzak, W. S., and Blanton, M. 2006. Platelet-rich plasma: a review of biology and applications in plastic surgery. Plasticand Reconstructive Surgery 118:147e-159e. 38-25 2012-05-29

4. Marx, R. E. 2001. Platelet-rich plasma (PRP): what is PRP and what is not PRP Implant Dent 10:225-228.

5. Everts, P. A., Knape, J. T., Weibrich, G., Schonberger, J. P., Hoffmann, J., Overdevest, E. P., Box, H. A., and van Zundert, A. 2006. Platelet-rich plasma and platelet gel: a review. J Extra Corpor Technol 38:174-187.

6. Kasten, P., Vogel, J., Geiger, F., Niemeyer, P., Luginbuhl, R., and Szalay, K. 2008. The effect of platelet-rich plasma on healing in critical-size long-bone defects. Biomaterials 29:3983-3992.

7. Akeda, K., An, H., Okuma, M., Attawia, M., Miyamoto, K., Thonar, E., Lenz, M., Sah, R., and Masuda, K. 2006. Platelet-rich plasma stimulates porcine articular chondrocyte proliferation and matrix biosynthesis. Osteoarthritisand Cartilage 14:1272-1280.

8. de Mos, M., van der Windt, A. E., Jahr, H., van Schie, H. T. M., Weinans, H., Verhaar, J. A. N., and van Osch, G. J. V. M. 2008. Can Platelet-Rich Plasma Enhance Tendon Repair: A Cell Culture Study. The American Journal of Sports Medicine 36:1171-1178.

9. Murray, M. M., Spindler, K. P., Ballard, P., Welch, T. P., Zurakowski, D., and Nanney, L. B. 2007. Enhanced histologic repair in a central wound in the anterior cruciate ligament with a collagenplatelet-rich plasma scaffold. Journal of Orthopaedic Research 25:1007-1017.

10. Harmon, K. G. 2010. Muscle injuries and PRP: what does the science say, British Journal of Sports Medicine 44:616-617.

11. Mei-Dan, O., Mann, G., and Maffulli, N. 2010. Platelet-rich plasma: any substance into it, British Journal of Sports Medicine 44:618-619.

12. Randelli, P. S., Arrigoni, P., Cabitza, P., Volpi, P., and Maffulli, N. 2008. Autologous platelet rich plasma for arthroscopic rotator cuff repair. A pilot study. Disability & Rehabilitation 30:1584-1589.

13. Song, H., Song, B. W., Cha, M. J., Choi, I. G., and Hwang, K. C. 2010. Modification of mesenchymal stem cells for cardiac regeneration. Expert Opin Biol Ther 10:309-319.

14. Karp, J. M., and Leng Teo, G. S. 2009. Mesenchymal stem cell homing: the devil is in the details. Cell Stem Cell 4:206-216.

15. Brenner, S., Whiting-Theobald, N., Kawai, T., Linton, G. F., Rudikoff, A. G., Choi, U., Ryser, M. F., Murphy, P. M., Sechler, J. M., and Malech, H. L. 2004. CXCR4-transgene expression significantly improves marrow engraftment of cultured hematopoietic stem cells. Stem Cells 22:1128-1133.

16. Cheng, Z., Ou, L., Zhou, X., Li, F., Jia, X., Zhang, Y., Liu, X., Li, Y., Ward, C. A., Melo, L. G., et al. 2008. Targeted migration of mesenchymal stem cells modified with CXCR4 gene to infarcted myocardium improves cardiac performance. Mol Ther 16:571-579.

17. Sackstein, R., Merzaban, J. S., Cain, D. W., Dagia, N. M., Spencer, J. A., Lin, C. P., and Wohlgemuth, R. 2008. Ex vivo glycan engineering of CD44 programs human multipotent mesenchymal stromal cell trafficking to bone. Nat Med 14:181-187.

18. Lee, R. J., Fang, Q., Davol, P. A., Gu, Y., Sievers, R. E., Grabert, R. C., Gall, J. M., Tsang, E., Yee, M. S., Fok, H., et al. 2007. Antibody targeting of stem cells to infarcted myocardium. Stem Cells 25:712-717.

19. Chavakis, E., Urbich, C., and Dimmeler, S. 2008. Homing and engraftment of progenitor cells: a prerequisite for cell therapy. J Mol Cell Cardiol 45:514-522.

20. Jo, C. H., Kim, J. E., Yoon, K. S., Lee, J. H., Kang, S. B., Han, H. S., Rhee, S. H., and Shin, S. 2011. Does platelet-rich plasma accelerate recovery after rotator cuff repair. A prospective cohort study. Am J Sports Med 39:2082-2090.

21. Jo, C. H., Kim, E. M., Ahn, H. J., Kim, H. J., Seong, S. C., and Lee, M. C. 2006. Degree of degeneration and chondroitinase ABC treatment of human articular cartilage affect adhesion of chondrocytes. Tissue Eng 12:167-176.

22. Brittberg, M., and Peterson, L. 1998. Introduction of an articular cartilage classifiction. ICRS News letter 1:5-8.

1. A pharmaceutical composition for increasing attachment and homing of cells comprising PRP (Platelet Rich Plasma).

2. The composition of claim 1, wherein the PRP comprises an amount of 2×10⁵ to 3×10⁸ platelets per microliter of plasma.

3. The composition of claim 1, wherein the cells are stem cells.

4. The composition of claim 3, wherein the stem cells are multipotent stem cells.

5. The composition of claim 4, wherein the multipotent stem cells are multipotent mesenchymal stem cells, multipotent hematopoietic stem cells, multipotent neural stem cells, multipotent endothelial stem cells, multipotent epithelial stem cells, multipotent epidermal stem cells, induced pluripotent stem cells, multipotent muscle stem cells, multipotent adult stem cells, multipotent pancreatic stem cells, multipotent cardiac stem cells, multipotent vascular stem cells, multipotent follicular stem cells, multipotent corneal stem cells, multipotent adipose stem cells, multipotent umbilical cord/umbilical cord blood stem cells, multipotent tooth stem cells, multipotent liver stem cells, or multipotent cancer stem cells.

6. The composition of claim 1, wherein the composition is used to pretreat ex vivo isolated cells in order to improve the cell's abilities for attachment and homing.

7. The composition of claim 1, wherein the composition is applied to an in vivo site to which the cells have been or are to be transplanted.

8. The composition of claim 1, wherein the composition is applied to an in vivo site in a mixture with the cells to be transplanted.

9. The composition of claim 1, wherein the cells have an increased homing activity to a damaged site.

10. A method for increasing cells' abilities for attachment and homing, the method comprising a step of treating isolated cells and/or in vivo site to which the cells are to be transplanted with the composition of claim 1.

11. A platelet-rich plasma for increasing cell's abilities for attachment and homing.
